(19) United States
(12) Patent Application Publication
(10) Pub. No.: US 2005/0053637 A1
(43) Pub. Date: Mar. 10, 2005

(54) Personalized cosmetics

(76) Inventors: Ze'ev Ma'Or, Kalia (IL); Yoram Milner, Mevaseret Zion (IL); Francois Menachem Bregeger, Jerusalem (IL)

Correspondence Address: DANIEL J SWIRSKY, PO BOX 2345, BEIT SHEMESH, 99544, IL

(21) Appl. No.: 10/934,441

(22) Filed: Sep. 7, 2004

(30) Foreign Application Priority Data
Sep. 7, 2003 (IL) IL 157786

(51) Int. Cl. A61K 38/00; G01N 33/53; G01N 33/567; A61K 31/535; A61K 7/00

(52) U.S. Cl. 424/401; 514/2; 435/7.2

(57) ABSTRACT

The present invention discloses a method and means for providing specific dermocosmetic compositions designed to correspond to individual skin condition. The method comprises inter alia the steps of sampling the skin to be treated, so as to allow the analysis of defined sets of biomarkers relevant to the pathology under treatment; measuring quantitatively each biomarker in said sampled skin, so as to determine an individual profile of selected biomarkers, which defines the individual skin condition; analyzing the biomarker profile and designing a skin care protocol especially adapted to said individual skin condition; administrating a plurality of dermo-cosmetic compositions, in accordance with said individually adapted skin care protocol; wherein a personal correlation between said sampled patient's skin and said administrated treatment is provided.

### FIELD OF THE INVENTION

The present invention generally relates to personalized cosmetics, i.e., to a method for providing specific dermo-cosmetic compositions designed to address individual skin conditions and to a kit providing the same.

### BACKGROUND OF THE INVENTION

Skin is the largest organ of the body and serves several vital functions, including body protection, absorption, secretion, excretion, thermo-regulation, pigment synthesis, sensory perception and immunity. It is constantly exposed to harmful environments and extreme conditions, such as ultra violet radiation, urban pollution, industrial contamination, pathogenic attacks, allergenic challenges, carcinogenic substances etc. Hence, the skin regularly needs topical treatment, which is usually provided by dermo-cosmetics, i.e., cosmetic preparations and/or dermal pharmaceutics. The dermo-cosmetics are targeted to achieve effective skin protection or prevention of skin ailments, by eliminating or avoiding skin sensitivity responses, skin irritation, skin xerosis or dryness, sun burn or suntan, by controlling or delaying skin aging, appearance of wrinkles, decreased skin smoothness, decreased general appearance (e.g., loss of healthy glow etc).

Commercially available dermo-cosmetics comprise effective active agents, formulated in various substances (gels, creams, lotions, masks etc.) and are usually made of high quality ingredients. Nevertheless, it has been suggested in the literature that due to relatively poor delivery characteristics of dermo-cosmetics, only a small portion of their active agents actually target treated skin and reaches the cells. Moreover, some users are sensitive to dermo-cosmetics preparations and tend to develop signs of irritability and toxicity following exposure to those chemicals. Some users are reported as not affected by the dermal treatment at all.

Today, users themselves make the correlation between their skin condition and the kind of dermo-cosmetic composition they need as a prior step before treatment. Users usually chose the product themselves “off the shelf” according to their understanding. Such purchases are extensively affected both by ever-changing fashions and by exposure of users to advertisements. In some cases the user consults the employee who stands at the point of sale. Usually, this promotional-marketing worker is not an expert on skin conditions. In other cases, cosmeticians examine the skin to be treated visually and subsequently, according to their accumulated experience, knowledge, and/or business orientation, suggest a recommended treatment. More rarely, a physician e.g., a dermatologist studies the topical appearance of the skin and gives professional advice to the user on the treatment.

It was stated before that most available dermal tests are based on human appraisal and statistics, rather than on cell biology. Furthermore, scientific examination of the skin involves a large number of expensive tests, e.g., toxicological or irritation tests, sensitization tests etc, which inhibit its incorporation into everyday mass practice. Consequently, it is rarely used unless the patient really suffers from serious skin problems.

Various skin-sampling techniques were disclosed in the literature. A dermatological punch biopsy is usually performed by means of a round knife ranging from 2 to 10 mm in size. A plurality of 5 mm punches generally provides adequate epidermis samples for analysis (See for example Zuber, T. J., 2002. Am. Fam. Physician 65, 1155-1164). Another epidermal sampling is denoted as ‘tape stripping’ and was introduced by Mattin et al., 1996. Skin Pharmacol. 9, 69-77, and by others. Suction blistering is an ex vivo sampling technique that was suggested by Falabella, R., 2000. Int. J. of Dermatol. 39, 670-672. According to this method, metal cups are attached to the skin surface of each forearm. Skin is suctioned using a vacuum pump with negative pressure of 150 mm Hg. Epidermal blisters, 1.6 cm in diameter, are induced after 2-5 hours of suction. Further epidermis sampling methods were suggested by Dimri, G. P. et al., 1995, Proc. Natl. Acad. Sci USA 92, 9363-9367 and by others.

Few molecular approaches were suggested in the art for diagnosing or treating skin. Among those suggested, U.S. Pat. No. 2,002,0034741 to Werner teaches the use of polypeptides or nucleic acids, encoded to create a gene family for the diagnosis or treatment of skin or intestinal disorders, and their use for the identification of pharmacologically active substances.

Similarly, U.S. Pat. No. 2,002,0012927 to Burmer discloses nucleic acids and proteins such as cell proliferation and senescence, associated with the aging process, and in particular with skin aging. This patent claims that a cosmetic composition containing those nucleic acids and proteins inhibits skin cell aging in a patient.

It is clear that a cost effective ex vivo dermal analysis, adapted to screen current skin condition and to provide a scientifically proven correlation between said skin condition and the most effective treatment by a dermocosmetic composition is a long-felt need.

### SUMMARY OF THE INVENTION

It is thus the main object of the invention to provide an efficient method for providing specific dermocosmetic compositions designed to correspond to individual skin conditions.

This method provides for a personal correlation between said patient's sampled skin and said administrated treatment, and comprises inter alia the following four steps: (a) sampling the skin to be treated, so as to allow the analysis of defined sets of biomarkers relevant to the pathology under treatment; (b) measuring each biomarker in said sampled skin quantitatively, so as to determine an individual profile of selected biomarkers, which defines the individual skin condition; (c) analyzing the biomarker profile and designing a skin care protocol especially adapted to said individual skin condition; and subsequently (d), administrating a plurality of dermocosmetic compositions, in accordance with said individually adapted skin care protocol.

It is in the scope of the present invention wherein the aforementioned skin condition is pathological and/or physiological. Pathologic conditions may include atopic, psoriasis, seborrheic, ichtiotic, acne, xerotic, irritation, allergy, or any combination thereof. This skin pathology may require local skin care. Physiological conditions may include ageing, dryness, hyperesthesic, hyperpilose, baldness, poor cicatrizing, low-pH, physiological conditions calling for local skin care or any combination thereof.

It is also in the scope of the present invention wherein the aforementioned skin sampling method is selected from punch biopsy, scraping, tape stripping, suction blister, or any recognized method adapted to take minute epidermis samples for analysis. The above-mentioned quantitative measurement is preferably performed through the analysis of RNA transcripts and/or their complementary DNA on high-density or low-density cDNA micro-arrays (i.e., bio-chips). Alternatively or additionally, the quantitative measurement of protein and/or protein-associated biomarkers is performed through biochemical or immunochemical analysis of epidermal soluble extracts. Preferably, the quantitative measurement is provided by means selected from ELISA tests, 1-D or 2-D electrophoresis, western blotting or any combination thereof.

Alternatively or additionally, the quantitative measurement of protein and/or protein-associated biomarkers is performed by intracellular analysis of protein amounts.

It is acknowledged in this respect that the intracellular analysis is preferably provided by means selected from immuno-fluorescent labeling or other selective fluorescent staining in situ, using a flow-cytometric device or a microtitration plate fluorometer for detection and measurement.

It is also in the scope of the present invention wherein the quantitative measurement of enzymatic biomarkers is performed through intracellular measurements of enzyme activities. These intracellular measurements of enzyme activities are preferably provided by means selected from use of fluorogenic substrates, use of a flow-cytometric device or a microtitration plate fluorometer for detection and measurement.

According to one embodiment of the present invention, the biomarkers are directly involved in at least one pathway selected from cellular senescence, apoptosis, differentiation, or any other pathway related to cell ageing.

According to yet another embodiment of the present invention, the biomarkers are indirectly involved in at least one physiological condition selected from epidermal cells undergoing replicative senescence or organism ageing. Hence, these biomarkers are present at altered levels in epidermal cells undergoing replicative senescence or organism ageing. Their levels range widely because they are differentially expressed, or because they are accumulated upon ageing as a consequence of oxidative stress, decrease in protein turnover or any other cause.

It is thus in the scope of the present invention wherein the aforementioned undirected biomarkers are selected from Apo J (i.e., clusterin), Ki-67, carbonyl groups (i.e., protein oxidation), 4-hydroxy-2-nonenal (i.e., lipid peroxidation adduct in proteins), carboxymethyl-lysine or advanced glycated endproducts (i.e., at least two kinds of glycoxidation adducts in proteins), ubiquitin adducts on proteins, total amount of cellular proteins, or any combination thereof.

It is also in the scope of the present invention wherein enzymatic biomarkers are selected from β-galactosidase, proteasome chymotrypsin-like or postglutamyl-peptidase activities. Alternatively or additionally, the biomarkers involved in a senescence pathway may be selected from: p161NK4a; Cdk4; Cdk6; p19ARF; p53; MDM2; p21Cip1; PTEN; p27Kip1, PI 3-kinase their transcripts or any combination thereof. Alternatively or additionally, the biomarkers involved in apoptosis pathways are selected from: Fas, Fas L, FADD, activated caspase 8 (i.e., cleaved protein), p53, Bcl-2, Bax, Apaf-1, caspase 3 activity, annexin V-binding sites.

It is also in the scope of the present invention wherein enzymatic biomarkers involved in keratinocyte differentiation are selected from involucrin and cytokeratines.

According to one embodiment of the present invention, the aforementioned method for arriving at specific dermocosmetic compositions designed to correspond to individual skin condition is incorporated with other skin treatments. Those skin treatments are selected from, yet not limited to: UV irradiating, bathing, administrating wide range cosmetics, administrating pharmaceutical compositions topically or systematically, or any combination thereof.

It is a second object of the present invention to present a novel kit for arriving at specific dermocosmetic compositions designed to correspond to individual skin condition. Thus, a cost-effective kit, especially adapted to provide a personal correlation between said sampled patient's skin and said administrated treatment, is provided.

The kit contains inter alia the following four ingredients: (a) means for sampling the skin to be treated, so as to allow the analysis of defined sets of biomarkers relevant to the pathological or other skin condition under treatment; (b) means for measuring quantitatively each biomarker in said sampled skin, so as to determine an individual profile of selected biomarkers which defines the individual skin condition; (c) means for analyzing the biomarker profile and for designing a skin care protocol especially adapted to said individual skin condition; and (d), means for administrating a plurality of dermocosmetic compositions, in accordance with said individually adapted skin care protocol.

It is in the scope of the present invention wherein the skin sampling means are adapted for punch biopsy, scraping, tape stripping, suction blister or for any combination thereof. The measuring means are preferably adapted for detecting biomarkers by intracellular analysis and/or molecular biology methods. The aforementioned molecular biology method preferably comprises cDNA arrays, mRNA, biochips, gene-arrays, proteomic arrays or micro array technique, 1D or 2D electrophoresis method, Western blot, ELISA or any other molecular biology methods. The aforementioned intracellular analysis comprises flow cytometry, FACS technologies, microtitration plate-fluorometry, immunolabeling techniques, or any other methods for intracellular analysis.

It is in the scope of the present invention wherein the biomarkers are directly involved in senescence pathways; differential expression at senescence, apoptosis pathways or any combination thereof. The biomarkers directly involved in senescence pathways are preferably selected from p161NK4a; Cdk4; Cdk6; p19ARF; p53; MDM2; p21Cip1; PTEN; p27Kip1 or PI 3-kinase.

It is in the scope of the present invention wherein the biomarkers, characterized by a differential expression at senescence, are selected from clusterin (Apo J), proliferation marker Ki-67, proteasome 20 S or 19 S subunits or total protein cellular content. Additionally or alternatively, the biomarkers involved in apoptosis pathways are selected from Bcl-2; Bax or Apaf-1, and/or adapted for flow cytometry and/or micro-titration plates. The enzyme biomarkers which are adapted for intracellular assays are potentially selected from biomarkers comprising the following activities: β-galactosidase activity; proteasome chymotrypsin-like activity or postglutamyl-peptidase activity.

### DETAILED DESCRIPTION OF THE INVENTION

The following description is provided, alongside all chapters of the present invention, so as to enable any person, skilled in the art, to make use of said invention and sets forth the best modes contemplated by the inventor of carrying out this invention. Various modifications, however, will remain apparent to those skilled in the art, since the generic principles of the present invention have been defined specifically to provide a method for targeting dermocosmetic compositions to a patient's skin, especially a method comprising the steps of sampling said skin in such a manner that biomarkers of skin condition are obtained; determining the sampled biomarkers; analyzing said skin condition; and then administrating dermocosmetic compositions targeted to the skin to be treated. This method provides for personalized cosmetics, i.e., a method for providing specific dermocosmetic compositions designed to correspond to individual skin condition, and a kit providing the same. The present invention permits the administration of specifically developed skin products, which fit the specific skin status of a specific client, with high efficacy, and low potential toxicity risk.

Human skin types display many variations due to genetics, age, lifestyle, environment, seasons etc. The regular ways to differentiate cosmetic and dermocosmetic products by skin type (i.e.: dry skin, very dry, oily, normal and combination skin) or by skin condition (i.e.: atopic, psoriatic, seborrehic, ichtiotic, acne, sensitivity, irritation etc.) are highly limited and do not reflect the endless skin variability of real life.

Based on human genome data, and on novel biotechnological methods for skin analysis, this invention presents the opportunity to develop personalized cosmetics, wherein the dermo-cosmetic products are specially formulated from A to Z or slightly adapted to fit each patient's skin, in order to improve skin health and beauty.

This patent also proposes to use novel technologies to evaluate the biomarkers of various skin conditions including skin senescence, differentiation, apoptosis, dryness, barrier functioning and more. The skin status analysis is based on a skin sample taken from the patient by one of various methods.

The term ‘sampling’ is denoted in the present invention to mean any in-vivo or ex-vivo protocol enabling sampling of a small portion of the skin, skin layers, skin cells, etc, and comprising inter alia the following techniques of skin sampling, biomarkers sampling: suction blistering, punch biopsies, blade scraping, tape stripping, etc.

The term ‘determining’ is denoted in the present invention to mean any in vivo, ex vivo or in vitro techniques adapted to measure either the presence or the amount of sampled biomarkers.

According to one embodiment of the present invention, a plurality of laboratory tests is provided for biomarkers determination. These tests are selected, yet not limited to intracellular tests, including especially various techniques in the field of micro arrays, such as high-density and low-density cDNA arrays, biochip, gen-arrays, proteomic arrays etc. These tests may also include other methods of molecular and cellular biology, such as flow cytometry (e.g., FACS) and other technologies, 2D electrophoresis method, Western blot, ELISA and other immunological techniques. Those methods have been proved useful, reliable, reproducible and cost-effective for skin analysis and biomarker determination.

As an example of the steps to be taken, a three step procedure is presented: (a) sampling the skin in such a manner that biomarkers of various skin conditions are obtained; (b) determining the sampled biomarkers; and then (c) analyzing specific said skin condition as defined in the present invention. A plurality of skin sampling protocols is provided, and in particularly an epidermal sampling technique such as punch biopsy. The purpose of those ex vivo sampling techniques is to evaluate the effects of topical applications of various products on “cellular age” in epidermal cells. The evaluation is mainly provided by comparing transcriptional patterns of treated versus untreated portions of epidermis, or by comparing data from a later skin specimen with those from early ones. Additionally or alternatively, analysis is made of protein markers of ageing, and of age-related enzyme activities in epidermal samples from treated versus untreated skin portions, or from young versus old skin portions.

The dermatologic punch used in the present invention is a round knife ranging from 2 to 10 mm in size. A plurality of 5 mm punches generally provides adequate epidermis samples for analysis. The experiment was found to be especially effective in healthy volunteers (i.e., patients) ages 18-65, without skin diseases or chronic medication or drug use who were not participating in any other research. Before performing a punch biopsy, the skin tension lines are determined. Thus, the skin is stretched perpendicularly to these tension lines before the incision, and an elliptic wound is formed in such a manner that it can be closed with the help of normal skin tension without “dog ears”. After cleansing the skin, local anesthesia is achieved by intradermal injection. The punch is placed perpendicularly to the skin surface. After applying a gentle pressure, it is rotated back and forth while advancing on the hub. The edge of the specimen is then grasped with toothed forceps, or “scooped” out with the punch, so as not to crush the skin. If the underlying fatty tissue retains the base of the sample, iris scissors are used to snip the tissue free. Hemostasis is obtained by applying aseptic solution, gauze and pressure. Subsequently, the wound is closed by one or two sutures to avoid a depressed scar.

Biopsies of skin samples are stored in “RNA later” solution (Promega) before RNA extraction, or put in PBS and frozen for protein analysis by ELISA or western blots. Total RNA is extracted using the “SV Total RNA Isolation System” (Promega). PolyA+ messenger RNA can be extracted using Micro-FasTrack mRNA Isolation Kit (Invitrogen). Two to four 5 mm-diameter biopsies are hence taken from each forearm, corresponding to 0.2 cm2 of epidermis per punch biopsy. About 100,000 keratinocytes are obtained from each punch biopsy, mixed with a small number of dermal fibroblasts (up to 5%). The yield of total RNA is about 10 μg per punch biopsy (up to 80 μg per operation).

It is also in the scope of the present invention to provide another epidermal skin sampling protocol, namely tape-stripping. This protocol is useful for evaluation of the effects of topical applications of various products on “cellular age” in epidermal cells: analysis of protein markers of ageing, and of age-related enzyme activities, in treated versus untreated skin portions. Here again, healthy volunteers are selected. The tape-stripping experiments are performed on the flexor forearm. Adhesive tape is applied to the skin of the flexor forearm, pressed to the skin area with a roller and pulled off in one quick movement as described. The first tape strip is discarded. The next 5 to 20 tapes can be subjected to protein extraction for western blot analysis or enzyme assay. Wounds are dressed with special hydrocolloid dressing, and heal within one week with no residual scars. In this protocol, the extraction yields are low when compared with surgical methods, since only a minority of epidermal cells is removed from the skin. For a treated surface of 10 cm², yields of 100-200 μg of protein can be obtained, depending on the number of successive strips.

Alternatively, another epidermal sampling protocol was used, namely suction blisters technique. This technique is especially useful for analysis of transcriptional changes in the expression of ageing markers, in skin epidermis from young and old donors, using high-density cDNA micro-arrays. From this data, determination of a set of markers showing differential transcription patterns (“profiles”) is provided, especially useful for evaluation of the effects of topical applications of various products on “cellular age” in epidermal cells, carried out by comparing transcriptional patterns in treated versus untreated portions of the epidermis.

Accordingly, metal cups are attached to each forearm skin surface. Skin is suctioned using a vacuum pump with negative pressure of 150 mm Hg. Epidermal blisters, 1.6 cm in diameter, are induced after 2-5 hours of suction. Blister fluid is collected for protein analysis by ELISA or Western blots. Epidermis samples are cut out from blister roofs and immediately frozen in liquid nitrogen, or processed right away for RNA extraction using the “SV Total RNA Isolation System” (Promega). Total RNA can be analyzed using the “Affymetrix cDNA micro arrays” transcript screening system or by specific skin low-density gene-arrays. Alternatively, epidermis samples are processed for analysis of specific protein markers, protein modification, or enzyme activities. Wounds are dressed with special hydrocolloid dressing, and heal within 10 days with no residual scars. Two to four blisters are suctioned on each forearm. About 2 cm² of epidermis and about 106 cells, essentially keratinocytes, can be obtained from each blister. About 100 μg of RNA or 500 μg of protein may be extracted per blister.

It was found that the yields of the hereto-defined methods (namely punch biopsies, tape stripping and suctions blisters) retained for systematic sampling provide sufficient amounts of RNA or proteins for at least one test per patient (in the case of punch biopsies) or more. Tape stripping is restricted to the study of protein modifications. Suction blistering was found appropriate for all the purposes of this project, including immunofluorescence and enzyme assays in situ. Because punch biopsies are commonly practiced in most dermatology departments, this method provides a valuable alternative for obtaining RNA samples whenever dependence on a specialized hospital facility may be a limiting factor.

It is further acknowledged that epidermis sampling is needed for the analysis of age-sensitive RNA transcripts and protein biomarkers, in order to evaluate the biological effects of potential active anti-ageing components following topical application.

It is additionally acknowledged that by performing periodical skin sampling and analyzing RNA extracts from skin one may determine up or down levels in the transcriptions of some ageing gene biomarkers.

Punch biopsy is the most direct method, allowing immediate sampling of skin slices that can be processed for either RNA or protein extraction. However, this method is affected by several drawbacks: a. local anesthetic is necessary, and might interfere with the expression of short-lived transcripts; b. the wound is expected to leave a minor scar, making the other methods preferable whenever punched slices of skin are not specifically needed; and c. punched skin slices contain dermis, and keratinocyte separation is delicate because of the scarcity of the material. This inconvenience is limited for RNA sampling because the accompanying dermis is constituted mainly of extracellular matrix and can contribute only a minority of cells and of RNA (less than 10%); but the interference of dermis is more severe for protein sampling; therefore the use of punch biopsies has to be restricted to RNA analysis. For cosmetic purposes, it is important indeed that all tests address “upper skin layers” (i.e.: epidermis) only, and do not penetrate to sample deeper layers of the skin, in order to remain legally in the field of cosmetology.

Tape stripping is much less invasive than punch biopsy, leaving only superficial wounds that heal without residual scars. Even after 30 repeated strippings, only epidermal material is collected, but the cells are heterogeneous and mostly representative of upper layers. These are keratinocytes, more or less deeply engaged in terminal differentiation and cell death, with very low yields of transcripts. Were a transcriptional pattern to be detectable, it would represent terminal differentiation rather than ageing status. The same applies also to protein markers, except protein modifications, which are expected to be conserved in cellular proteins along the differentiation process. Tape stripping therefore seems to be the method of choice for the study of protein modifications in epidermis.

Skin scraping with a scalpel or razor blade resembles tape stripping, but is more traumatic, and inaccurate in the selection of cell layers. It has therefore not been considered.

Suction blistering offers the advantage of performing a clean separation between dermis at blister bottom and epidermis at blister roof. Epidermis comes off in its entirety including the basal layer. The procedure is almost painless, no anesthetic is used, and the wound is superficial and heals without a scar. Blister roof, when cut, can be processed directly either for RNA or for protein extraction. Blister fluid can be collected and used for protein analysis. Suction blistering appears therefore most appropriate for our purpose, yet has the drawback that suction itself has to be continued for 2 to 4 hours at ambient temperature, during which the marker profiles might be perturbed.

It is also in the scope of the present invention to use cellular markers for flow cytometry. Those non-immunologic, cellular markers are selected in a non-limiting manner from the group of biomarkers characterized by: β-galactosidase activity, preferably studied by means of an intracellular assay; proteasome chymotrypsin-like and postglutamyl-peptidase activities, preferably studied by means of an intracellular assay; total protein content, preferably studied by FITC labeling. Some of those markers are characterized by an expected up-regulation upon ageing, and others by down-regulation.

β-galactosidase activity was found to increase notably upon ageing in most mammalian tissues including epidermis. More specifically, it was proved that β-galactosidase activity increases by a factor 4 in human epidermis between the ages of 20 and 80.

It is also in the scope of the present invention wherein the aforementioned undirected biomarkers are selected from Apo J (i.e., clusterin), Ki-67, carbonyl groups e.g., (protein oxidation), 4-hydroxy-2-nonenal (i.e., lipid peroxidation adduct in proteins), carboxymethyl-lysine or advanced glycated endproducts (e.g., at least two kinds of glycoxidation adducts in proteins), ubiquitin adducts on proteins, total amount of cellular proteins or any combination thereof.

It is acknowledged in this respect that the amount of ApoJ transcripts (i.e., an mRNA) increases, upon replicative senescence, by a factor 11 in human osteoblasts and by a factor of 24 in rat embryonal fibroblasts. Total protein contents in human keratinocytes were found to increase by at least a factor of 5 upon replicative senescence.

It is also in the scope of the present invention wherein enzymatic biomarkers are selected from β-galactosidase, proteasome chymotrypsin-like or postglutamyl-peptidase activities. Alternatively or additionally, the biomarkers involved in a senescence pathway may be selected from: p161NK4a; Cdk4; Cdk6; p19ARF; p53; MD2; p21Cip1; PTEN; p27Kip1 or PI 3-kinase. Alternatively or additionally, the involved biomarkers in apoptosis pathways are selected from: Fas, Fas L, FADD, activated caspase 8 (i.e., cleaved protein), p53, Bcl-2, Bax, Apaf-1, caspase 3 activity, annexin V-binding sites.

The amounts of p161NK4a were found to increase by at least a factor of 2 upon replicative senescence in cultured human keratinocytes. The amounts of Fas receptor were found to increase by a factor of 4 in human epidermis between the ages of 20 and 80.

It is also in the scope of the present invention wherein enzymatic biomarkers involved in keratinocyte differentiation are selected from involucrin and from cytokeratines.

The proteasome chymotrypsin-like and postglutamyl-peptidase activities, in human epidermal extracts, were hereto proved to decrease by a factor of 3 between the age of 20 and the age of 60-70.

1. A method for providing specific dermocosmetic compositions designed to correspond to individual skin condition comprising:
a. sampling the skin to be treated, so as to allow the analysis of defined sets of biomarkers relevant to the pathology under treatment;
b. measuring quantitatively each biomarker in said sampled skin, so as to determine an individual profile of selected biomarkers, which defines the individual skin condition;
c. analyzing the biomarker profile and designing a skin care protocol especially adapted to said individual skin condition;
d. administrating a plurality of dermo-cosmetic compositions, in accordance with said individually adapted skin care protocol;
wherein a personal correlation between said sampled patient's skin and said administrated treatment is provided.

2. The method according to claim 1, wherein the skin condition is pathological and selected from atopic, psoriatic, seborrheic, ichtiotic, acne, xerotic, irritation, allergy or any combination thereof.

3. The method according to claim 1, wherein the skin condition is pathological and selected from pathologic conditions requiring local skin care.

4. The method according to claim 1, wherein the skin condition is physiological and selected from: ageing, dryness, hyperesthesic, hyperpilose, baldness, poor cicatrizing, low-pH, physiologic condition calling for local skin care or any combination thereof.

5. The method according to claim 1, wherein the skin sampling method is selected from punch biopsy, scraping, tape stripping, suction blister, or any recognized method adapted to take minute epidermis samples for analysis.

6. The method according to claim 1, wherein the quantitative measurement of biomarkers is performed through the analysis of RNA transcripts and/or their complementary DNA on high-density or low-density cDNA microarrays (bio-chips).

7. The method according to claim 1, wherein the quantitative measurement of protein and/or protein-associated biomarkers is performed through biochemical or immunochemical analysis of epidermal soluble extracts.

8. The method according to claim 7, wherein the quantitative measurement is provided by means selected from ELISA tests, 1-D or 2-D electrophoresis, western blotting or any combination thereof.

9. The method according to claim 1, wherein the quantitative measurement of protein and/or protein-associated biomarkers is performed by intracellular analysis of protein amounts.

10. The method according to claim 9, wherein the intracellular analysis is provided by means selected from immunofluorescent labeling or other selective fluorescent staining in situ, using a flow-cytometric device or a microtitration plate fluorometer for detection and measurement.

11. The method according to claim 1, wherein the quantitative measurement of enzymatic biomarkers is performed through intracellular measurements of enzyme activity.

12. The method according to claim 11, wherein intracellular measurements of enzyme activities are provided by means selected from use of fluorogenic substrates, use of a flow-cytometric device or a microtitration plate fluorometer for detection and measurement.

13. The method according to claim 1, wherein the biomarkers are directly involved in at least one pathway selected from cellular senescence, apoptosis, differentiation, or any other pathway related to cell ageing.

14. The method according to claim 1, wherein the biomarkers are indirectly involved in at least one physiological condition selected from epidermal cells undergoing replicative senescence or organism ageing.

15. The method according to claim 14, wherein indirectly involved biomarkers are selected from Apo J (clusterin), Ki-67, carbonyl groups (protein oxidation), 4-hydroxy-2-nonenal (lipid peroxidation adduct in proteins), carboxymethyl-lysine or advanced glycated endproducts (two kinds of glycoxidation adducts in proteins), ubiquitin adducts on proteins, total amount of cellular proteins or any combination thereof.

16. The method according to claim 11, wherein enzymatic biomarkers are selected from β-galactosidase, proteasome chymotrypsin-like or postglutamyl-peptidase activity.

17. The method according to claim 13, wherein biomarkers involved in a senescence pathway are selected from: p16INK4a; Cdk4; Cdk6; p19ARF; p53; MDM2; p21Cip1; PTEN; p27Kip1 or PI 3-kinase.

18. The method according to claim 13, wherein biomarkers involved in apoptosis pathways are selected from: Fas, Fas L, FADD, activated caspase 8 (cleaved protein), p53, Bcl-2, Bax, Apaf-1, caspase 3 activity, or annexin V-binding sites.

19. The method according to claim 13, wherein biomarkers involved in keratinocyte differentiation are selected from involucrin and from cytokeratines.

20. The method according to claim 1, additionally comprising other skin treatments.

21. The method according to claim 20, wherein the additional skin treatment is selected from UV irradiation, bathing, administration of wide range cosmetics, administration of pharmaceutical compositions topically or systematically or any combination thereof.

22. A kit for providing specific dermocosmetic compositions designed to correspond to individual skin condition comprising inter alia:
a. means for sampling the skin to be treated, so as to allow analysis of defined sets of biomarkers relevant to the pathological or other skin condition under treatment;
b. means for measuring quantitatively each biomarker in said sampled skin, so as to determine an individual profile of selected biomarkers, which defines the individual skin condition;
c. means for analyzing the biomarker profile and designing a skin care protocol especially adapted to said individual skin condition; and,
d. means for administrating a plurality of dermocosmetic compositions, in accordance with said individually adapted skin care protocol;
wherein a personal correlation between said sampled patient's skin and said administrated treatment is provided.

23. The kit according to claim 22, wherein the skin sampling means are adapted for punch biopsy, scraping, tape stripping, suction blister or for any combination thereof.

24. The kit according to claim 22, wherein the measuring means is adapted for detecting biomarkers using intracellular and/or molecular biological methods.

25. The kit according to claim 24, wherein the molecular biology analysis comprises cDNA arrays, mRNA, bio-chips, gene-arrays, proteomic arrays or micro array technique, 1D or 2D electrophoresis method, Western blot, ELISA, or any other molecular biological methods.

26. The kit according to claim 24, wherein the intracellular analysis comprises flow cytometry, FACS technologies, microtitration plate-fluorometry, immunolabeling techniques, or any other method for intracellular analysis.

27. The kit according to claim 22 wherein the biomarkers are directly involved in senescence pathways; differential expression at senescence; apoptosis pathways or any combination thereof.

28. The kit according to claim 27, wherein the biomarkers directly involved in senescence pathways are selected from p161NK4a; Cdk4; Cdk6; p19ARF; p53; MD2; p21Cip1; PTEN; p27Kip1 or PI 3-kinase.

29. The kit according to claim 27, wherein the biomarkers characterized by a differential expression at senescence are selected from clusterin (Apo J); proliferation marker Ki-67; proteasome 20 S or 19 S subunits, or total protein cellular content.

30. The kit according to claim 27, wherein the biomarkers involved in apoptosis pathways are selected from Bcl-2; Bax or Apaf-1.

31. The kit according to claim 27, wherein the biomarkers are adapted for flow cytometry and/or microtitration plate-fluoronetry.

32. The kit according to claim 31, wherein the biomarkers adapted for flow cytometry are selected from biomarkers comprising the following activities: β-galactosidase activity; proteasome chymotrypsin-like activity or postglutamyl-peptidase activity.
